(19) United States
(12) Patent Application Publication
(10) Pub. No.: US 2008/0008620 A1
(43) Pub. Date: Jan. 10, 2008

(54) Bimodal light bulb and devices for sterilizing and cleansing

(76) Inventor: Alkis Alexiadis, Wellington, FL (US)

Correspondence Address: Dara L. Onofrio, Esq.;c/o ONOFRIO LAW, 107 Shad Row, Piermont, NY, 10968, US

(21) Appl. No.: 11/821,309

(22) Filed: Jun. 22, 2007

Related U.S. Application Data
(60) Provisional application No. 60/816,195, filed on Jun. 23, 2006.

(51) Int. Cl. A61L 2/10; F21S 15/00; F21S 4/00

(52) U.S. Cl. 422/24; 362/231; 362/362; 422/186.3

(57) ABSTRACT

A light device comprised of an ultraviolet light source and an additional light source; wherein the additional light source is selected from the group consisting of a white light fluorescent and incandescent light; and a related system for sterilizing and cleansing a surface comprising the light device and a switch selected from the group consisting of infrared, movement, electric eye, manual, contact switches, auto or semi-automatic, hard wired, pre- and after market versions; wherein the switch controls the activation and deactivation of the ultraviolet light source and the additional light source such that they are not both on at the same time.

This application claims the benefit of U.S. provisional application no. 60/816,195 filed Jun. 23, 2006, which is incorporated herein in its entirety by reference.

### FIELD OF THE INVENTION

The present invention relates to a bimodal light bulb including an ultraviolet light source and a fluorescent or incandescent light source. In particular, the invention is a sterilizing and cleansing device which is controlled by an on/off switch with a movement sensor.

### BACKGROUND OF THE INVENTION

During the past century with the development of technology and growth of mechanical and chemical industries numerous pollutants and toxins have been released into our environment. It has been recently reported that for every pound of toxic chemicals released into the environment, 42 pounds of toxic chemicals were shipped into American homes on food as well as consumer products.

Chemical residues cover our interior and exterior home surfaces and fill the air we breathe. A typical family incurs daily exposure to many of these toxins through the mouth, skin and lungs.

It is a belief of many medical doctors and scientists that there is a strong link between the increased presence of toxic chemicals in the home and the concurrent increased incidence of cancer, asthma, neurological disease and birth defects. Children are up to ten times more vulnerable to chemical pollutants and the elderly are more vulnerable and susceptible to germs and chemicals.

Additionally, more than 76 million cases of food poisoning are reported in the United States each year. Of the $137 billion in produce consumed, more than 98% of it is contaminated with germs, chemicals and pesticides.

Hostile debilitating germs exit in our environment. It is known that the ultraviolet (UV) spectrum light provides an antiseptic field wherever it casts upon. It kills both germs and bacteria. It creates a hostile environment for germs and bacteria so that they can't grow or thrive in its' presence. It prohibits, retards, and prevents harmful mold growth. It provides a hostile environment for dust mites in carpets and upon linens, thereby reducing allergens and irritants and allergic reactions and asthmatic exacerbations.

In general light bulb and devices using either fluorescent/incandescent or ultraviolet light sources are well known. Representative of such devices are disclosed in the following patents and publications: U.S. Pat. No. 6,965,205 to Piepgras et al.; U.S. Pat. No. 6,953,265 to Suehiro et al.; U.S. Pat. No. 6,685,852 to Setlur et al.; U.S. Pat. No. 6,428,756 to Barnes; U.S. Pat. No. 5,904,415 to Robertson et al.; U.S. Pat. No. 5,477,054 to Tateosian et al.; U.S. Pat. No. 4,754,194 to Feliciano et al. and U.S. Publication No. 2002/0167263 to Nishio et al.

There are also a number of devices known in the art that include ultraviolet light sources as well as a white light source as well as the use of ultraviolet light as a germicidal device. Representative patents and publications of such devices include U.S. Pat. No. 7,018,08 to Hopkins; U.S. Pat. No. 6,589,323 to Korin; U.S. Pat. No. 6,497,840 to Palestro et al.; U.S. Pat. No. 5,958,336 to Duarte; U.S. Pat. No. 5,866,076 to Fencl et al.; and Publication No. 2003/0217641 to Palestro et al.

U.S. Patent Publication No. 2004/0252499 A1 to Tumlinson et al. which discloses a lamp assembly where the user can manually switch between a white light source and an ultraviolet light source. However, unlike the present invention, the light source itself faces upward toward a ceiling and there is no mention of the germicidal properties of the light sources.

U.S. Pat. No. 5,664,867 to Martin et al. which discloses a night light for a toilet seat. The light consists of a white light which is activated when the toilet seat is lifted. There is no mention of a ultraviolet light source or germicidal properties of the light sources.

In general, ultraviolet radiation is used in hospitals across the country to aid in sterilization of surfaces. Ultraviolet radiation is accepted by the FDA and USDA as a safe and effective way to decontaminate the environment and foods.

None of the devices, or uses, known in the prior art disclose a bimodal light device consisting of an ultraviolet light source and a fluorescent or incandescent light source for sterilizing and cleansing exposed surfaces. The light device which is part of a system can be controlled by an on/off switch or movement sensors on the device itself or as separate controls. When the movement sensor is activated the fluorescent/incandescent light source is on and when no movement is detected the ultraviolet light source is activated sterilizing and cleansing germs and bacteria on the surrounding area surfaces.

The present invention provides the opportunity to reduce one's exposure to harmful pathogens, bacteria and germs without introducing any chemicals at all.

The invention device is also able to extend food shelf life by keeping it fresher, nutritious and appetizing longer and saves money in the process.

The device reduces the need for chemical sanitizers, thus helping reduce the amount of chemical toxins released into the environment and home. The device disinfects all surfaces on contact, including kitchen counters, stovetops, sinks, bathrooms, toilets and garbage areas. It also produces a hostile environment for dust mites, a known trigger of asthmatic exacerbations. The device reduces and prevents mold and mildew growth on walls, in basements and attics. It helps neutralize odors in carpets, closets, clothes, upholstery and furniture. The device even sterilizes and cleans the air we breathe.

Play areas, toys, pacifiers, utensils, sponges and kitchen gadgets can be sanitized without harmful chemicals and residues.

The invention device is effective and is natural and safe. It provides a germicidal and bacteriacidal cleansing of everything it casts its light upon.

Every house, especially the bathroom and kitchen are constant breeding grounds for harmful bacteria and infectious germs. Bathrooms in particular receive a continuous misting of harmful germs with every flush of the toilet. When the toilet is flushed a mushroom cloud of micro-mist particles containing E coli bacteria bathes and covers every surface in the bathroom. This includes toothbrushes, razors, hairbrushes and all exposed surfaces. If these surfaces were swabbed with a Q-tip one would find the same harmful germs they find in their toilets. An individual would not think of dipping their toothbrush or hairbrush in the toilet before using it, but these germs in your toilet are already on these very personal items, renewed by each flush.

The kitchen is also a breeding ground for bacteria and germs and E. coli that cause colds, influenza and sickness, aggravate asthmatic conditions and cause illness and malaise. These germs are shared and spread by the hundreds of hands touching the packages an individual purchases, which in turn are touched by the hundreds of other hands even before they were purchased and then placed on the your counters and shelves. Items, bags, containers, satchels are placed on the floors of malls, shops, restaurants, car mats and then placed on the same counters and surfaces that your hands touch, your clothes contact and your food is prepared upon. Individuals walk around all day on the same surface that people sneeze, spit and cough upon, that animals urinate and defecate upon. All these germs are brought into one's home on shoes and clothing, spreading them around the home and in the environment.

The present solution to this toxic onslaught is to bring in more harmful and noxious chemicals in an attempt to cleanse the home. These materials are noxious, toxic and some containing cancer causing agents which are not effective in killing the biological and bacteriological invaders brought into the home. Instead of getting rid of these germs these material add to the other non-biological pollutants. These germs, bacteria and chemicals cause internal damage to every system in one's body.

A general objective of the invention is to provide a continual anti-microbial, germicidal, anti-bacterial clean sweep of any area in which it is placed.

Another object of the invention is to negate and limit the need to use harmful industrial chemicals in an individual's home environments.

A further object of the invention is to reduces germs on surface thereby reducing the onset of common illnesses and their transference.

Yet another object of the invention is to clean the air and reduce and eradicate air borne bacteria, which are often the cause of childhood and adult asthmatic exacerbations.

Still a further object of the invention is to provide a safer, healthier and cleaner living environment allowing people to thrive more easily.

Another object of the invention is to reduce that incidence of illnesses, colds, the flu and their subsequent malaise.

A more specific object of the invention is in continued use of the invention device which is believed to improve an individual's health and to reduce medical visits and related costs.

Another object to the invention is to provide a home with supportive healthier areas.

Another object of the invention device is that it works continuously and is quiet, efficient, effective and inexpensive.

Another object of the invention is to provide a hostile environment to germs and bacteria while providing an individual with a safer, cleaner and healthier habitat.

### SUMMARY OF THE INVENTION

In the present invention, these purposes, as well as others which will be apparent, are achieved generally by providing a light bulb comprised of an ultraviolet light filament and a white light filament contained within a single outer covering.

The invention also provides a light device made of an ultraviolet light source and an additional light source; wherein the additional light source is selected from the group consisting of a white light fluorescent and incandescent light.

The light bulb and device, preferably further includes a motion sensor. When movement is detected the sensor turns off the ultraviolet light source and turns on the additional light source. When movement stops the sensor turns off the additional light source and turns on the ultraviolet light source sterilizing and cleansing the surface in view. The motion sensor may be on the device itself or outside the device on a wall or door.

The invention also includes a process for sterilizing and cleansing a surface by providing a light device comprised of an ultraviolet light source and an additional light source; wherein the additional light source is selected from the group consisting of a white light fluorescent and incandescent light. The ultraviolet light source is activated to sterilize and cleanse the surface in view, while the additional light source is deactivated. When the ultraviolet light source is deactivated, the additional light source may be activated for conventional uses.

The activation and deactivation of the light sources are controlled by a switch which is selected from the group consisting of electric eye, infrared, a movement sensor, manual, automatic, hardwired, pre- or semi-automatic and after market. In the various embodiments described herein at least one switch is used. In alternate embodiments multiple switches can be used in the invention systems.

In general the invention system provides for sterilizing and cleansing a surface using a light device comprised of an ultraviolet light source and an additional light source; wherein the additional light source is selected from the group consisting of a white light fluorescent and incandescent light; and a switch selected from the group consisting of infrared, movement, electric eye, manual, contact switches, auto or semi-automatic, hard wired, pre- and after market versions. The switch controls the activation and deactivation of the ultraviolet light source and the additional light source such that they are not both on at the same time. The switch also provides that the ultraviolet light source is not activated in the presence of an individual.

In an alternate embodiment the invention device is controlled by an on/off switch. The fluorescent/incandescent light source is activated when an individual is present in the area illuminated. The ultraviolet light source is activated for sterilizing and cleansing germs and bacteria on the surrounding area surfaces when the fluorescent/incandescent light source is off.

The invention device encompasses various embodiments including a circular fluorescent or white light source with an inner portion having multiple ultraviolet or light emitting diode (LED) sources. Alternatively, the inner portion can be a circular ultraviolet light source. In yet another embodiment the inner portion can be an incandescent light bulb with an outer ring of an ultraviolet light source.

In all of the invention embodiments, with the exception of Embodiment VI, the light devices include at least one ultraviolet light source and at least one additional light source. In additional embodiments, multiple ultraviolet and additional light sources can be used depending on desired applications.

Embodiment VI describes a housing consisting of either a tape strip or rope where an ultraviolet light source is secured. This embodiment has preferred applications in attics, along framing and baseboards, in crawl spaces, etc. where it may not be necessary to include an additional light source. In an alternate embodiment, this housing may include a white light fluorescent and incandescent light which are controlled by switches described in the other invention embodiments.

In a preferred embodiment the activation of the light source is controlled by a motion sensor wherein each light source is activated by motion or lack thereof. In alternate embodiments an on/off switch controls which light source is activated.

Other objects, features and advantages of the present invention will be apparent when the detailed description of the preferred embodiments of the invention are considered with reference to the drawings, which should be construed in an illustrative and not limiting sense.

### BRIEF DESCRIPTION OF THE DRAWINGS

FIG. 1 is an illustration of the general features of the invention.

FIG. 2 illustrates various embodiments of the invention; specifically FIG. 2A is an illustration of Embodiment I of the invention device; FIG. 2B is an illustration of Embodiment II of the invention device; FIG. 2C is an illustration of Embodiment III of the invention device; FIG. 2D is an illustration of Embodiment IV of the invention device and FIG. 2E is an illustration of the system of the invention including the light device and respective housing.

FIG. 3 is an illustration of Embodiment V of the invention device;

FIG. 4A is an illustration of Embodiment VII of the invention device with switches in the housing; and FIG. 4B is an illustration of Embodiment VII of the invention device with motion sensors outside the device;

FIG. 5A illustrates a recessed lighting embodiment of the invention and FIGS. 5B and 5C are illustrations of the various housing devices for the invention;

FIGS. 6A to 6C illustrate the process and sterilizing and cleansing system of the invention; and

FIGS. 7A TO 7F illustrate Embodiment VI of the invention device showing the strip and rope housing.

### DETAILED DESCRIPTION OF THE INVENTION

In accordance with the present invention a light device comprised of an ultraviolet light source and an additional light source; wherein said additional light source is selected from the group consisting of a white light fluorescent and incandescent light is provided. As described in the accompanying drawings the structures of the invention device are labeled as follows:
1—white light filament
2—ultraviolet (UV) filament
3—housing
4—switches
5—fluorescent light
6—ultra violet light emitting diodes (UV LEDs)
7—white light
8—ultraviolet (UV) light
9—female socket/receptacle (optional)
11—ultraviolet fluorescent
12—incandescent
13—housing
14—male socket/receptacle
15—light cover
16—plastic or metal housing
17—UV light fluorescents
18—white light fluorescents
19—switches
42—extension switch with end sensors
51—white light
52—UV light
53—infrared heat sensor switch
54—movement switch
56—round light cover
57—opening for IR heat sensor
58—opening for movement switch
59—square light cover
61—contact switch
62—hard wire or wireless relay
63—UV light source
64—natural light source
65—white light
66—IR light switch
67—motion switch
68—UV LEDs
71—strip housing
72—ultraviolet light source
73—white light source
76—rope housing

As used in the invention herein the light source may include a light-emitting diode (LED). An LED is a semiconductor device that emits narrow-spectrum light when electrically biased in the forward direction of the p-n junction. This effect is a form of electroluminescence. An LED is a small area source with extra optics added to the chip that makes it emit a certain radiation pattern. The color of the emitted light depends on the composition and condition of the semi-conducting material used, and can be infrared, visible or near-ultraviolet.

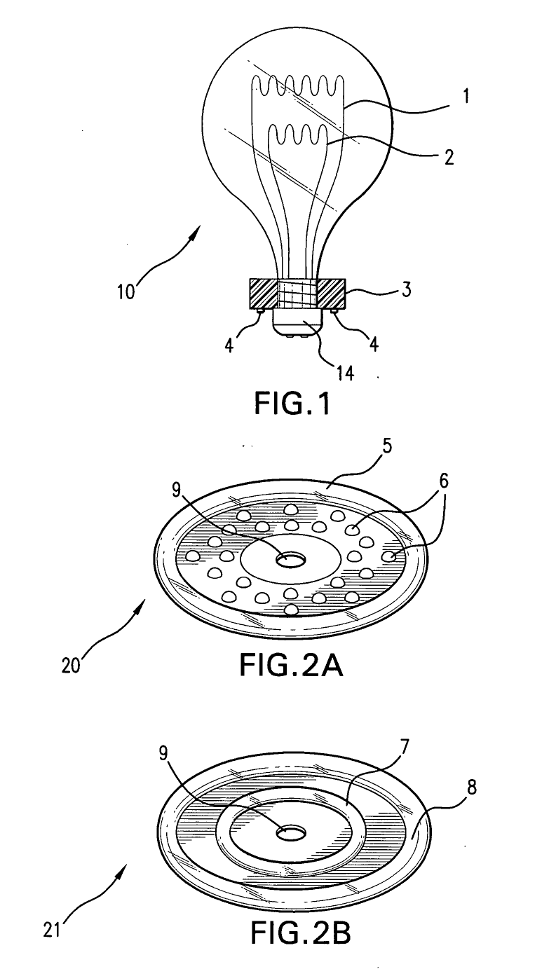

In general, the invention provides a bimodal light bulb. As illustrated in FIG. 1 the light bulb 10 is comprised of an ultraviolet filament 2 and a white light filament 1 within a single outer covering. Although the illustration shows the UV filament inside the white light filament their locations can be changed without changing the invention.

The bulb further includes a housing unit 3, which may include a switch 4. The switch may alternatively be outside of the housing unit, but in any event controls the activation of the UV and white light filaments such that only one filament is on at a time.

The invention also provides a light device comprised of an ultraviolet light source and an additional light source; wherein said additional light source is selected from the group consisting of a white light fluorescent and incandescent light.

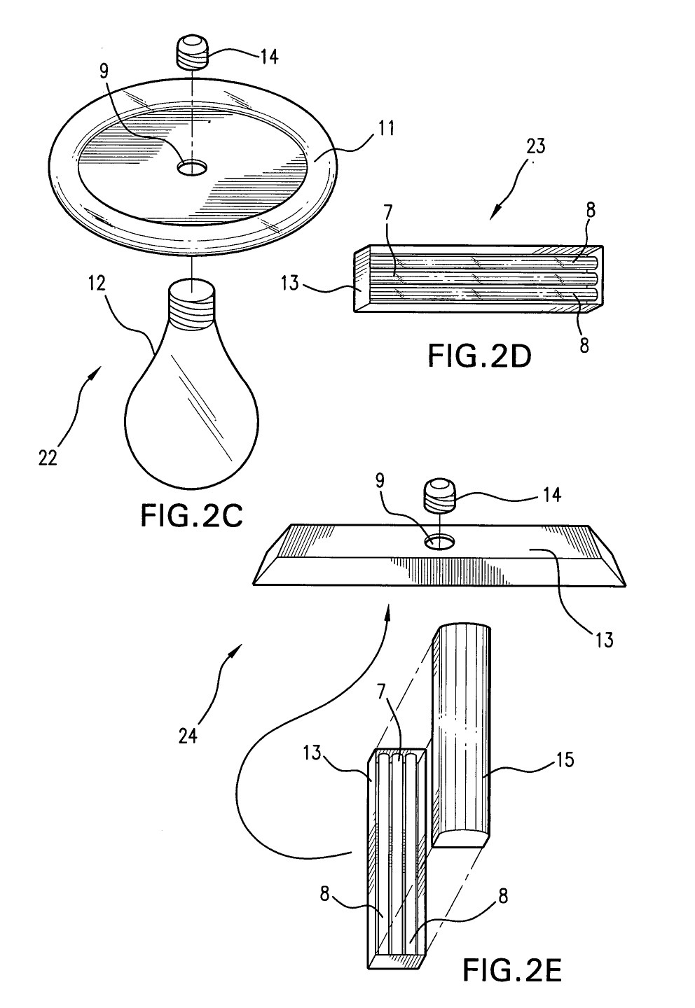

This device is used in a system for sterilizing and cleansing which includes an on/off switch or a movement sensor; a male screw to fit into a conventional female screw type socket; a housing unit itself for the light source (attached to or as an integral part of the housing unit); a ultraviolet light source either as LED's or as a fluorescent or incandescent light source, a female receptacle for attaching a normal fluorescent light source. FIG. 2C illustrates an outer ultraviolet fluorescent 11 light with an incandescent light 12 source in the center of the device 22.

The invention can be used in any conventional lighting socket. Typically, the conventional light source is removed. The invention device is attached by screwing it into, or placing it in the existing receptacle. In alternate embodiments, a white light source, incandescent or fluorescent light source is attached to the receptacle on the opposite side of the housing unit body. When the electric current is turned on the device light sources are activated. FIG. 2B illustrates an adapter or receptacle 9 to fit a fluorescent type bulb connector arrangement).

The invention provides a number of embodiments of the device. While the configurations may vary, the basic components, the modes of use and the underlying premises and sequencing of operation remain consistent. The figures aid in illustrating the invention embodiments but are not meant to be all encompassing due to the number of variations possible.

Embodiment I is illustrated in FIG. 2A and the system of FIG. 2E. The light device includes a white light source 7 (ring fluorescent or LED's); ultraviolet source LEDs 6; plastic or metal housing 13; a male socket component 14; and a switch 4.

The switches used in the invention can be on/off or delay, or a combination of both.

In a preferred embodiment, a movement sensor turns off the UV light source if a person enters the room or before the person enters that room as the door opens. The same switch turns on the white light source when movement is detected in the room as the door opens.

In another preferred embodiment an infrared switch turns on the UV light source if no body heat is detected n the room for a given period of time. The UV light is continually on or for specified time intervals. For example, if no heat is felt for 1 minute the UV light is activated. This activation can be continuous or for timed intervals depending on desired use and application.

In still another embodiment, a radio frequency contact switch can be placed at doors which turn off the ultraviolet light when contact is broken, as when opening a door. This is an additional layer of protection beyond those of the motion and infrared heat sensors themselves.

Configuration and interplay of which switches will act as master overrides may vary upon manufacturer and consumer preferences and needs.

Embodiment II is illustrated in FIG. 2B and the system of FIG. 2E. The light device includes a white light source 7 (ring fluorescent or LED's outer ring; an ultraviolet light source 8 (ring fluorescent or LED's inner ring); plastic or metal housing 13; male socket component 14. In an alternate version, where the device doesn't have male socket component, it is made ready for hard wiring. The location of the light sources, i.e. inner and outer rings can be interchanged without changing the invention. The switches used with this device are the same as described with Embodiment I.

Embodiment III is illustrated in FIG. 2C and the system of FIG. 2E. The light device includes a white light source 12 (center incandescent or LED) and an ultraviolet light source 11 (outer ring or LED's); plastic or metal housing 13; male socket component 14 (top) and female socket component 9 (bottom). The switches used with this device are the same as described with Embodiment I.

Embodiment IV is illustrated in FIG. 2D and the system of FIG. 2E. The light device includes a least three bulbs including a white light source 7 (2 or more long fluorescent or LEDs) and an ultraviolet light source 8 (one or more long fluorescent or LEDs). Alternatively there could be 2 or more UV lights and one or more white lights. Plastic or metal housing 13; male socket component 14 (top or hard wired option); female socket component 9 (bottom) and decorative plastic cover 15 for housing 13 are shown in FIG. 2E, The switches used with this device are the same as described with Embodiment I.

In all embodiments, the ultraviolet or white light sources may be interchanged and may also be replaced with LEDs for ease of configuration and design.

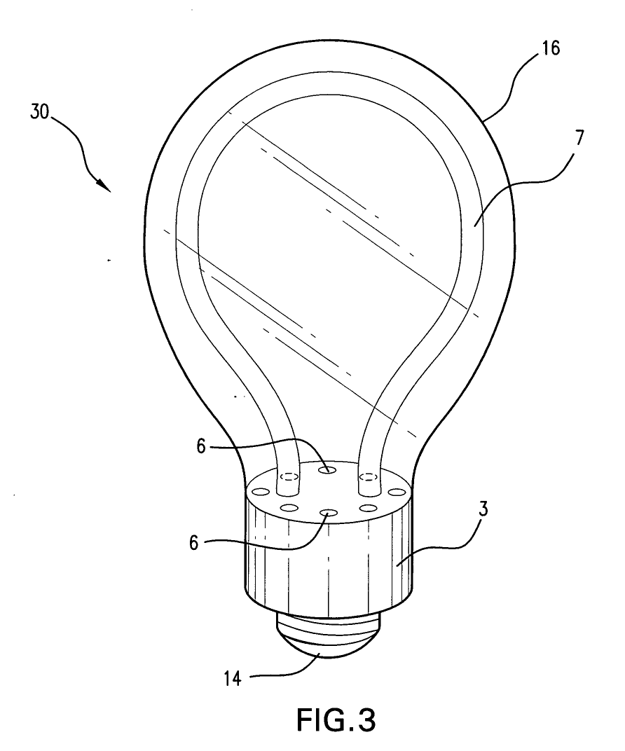

Embodiment V is illustrated in FIG. 3. A preferred application is use of this light device in a refrigerator. The light device includes a white light 7 source (refrigerator bulb) and an ultraviolet light source 6—LEDs in the base of the bulb housing 3, in a ring around the perimeter of the bulb housing; plastic or metal housing 16; and a male socket component 14.

In this embodiment, the light source is hard wired. When the refrigerator door is open the white light source is on, when the refrigerator door is closed the ultraviolet LEDs is activated and acts as a bacteriacidal/germicidal light source thereby killing and further retarding bacterial growth, prolonging the freshness and use of foodstuffs beyond the effectiveness of refrigeration alone. The device is designed to fit in all types of refrigerator models.

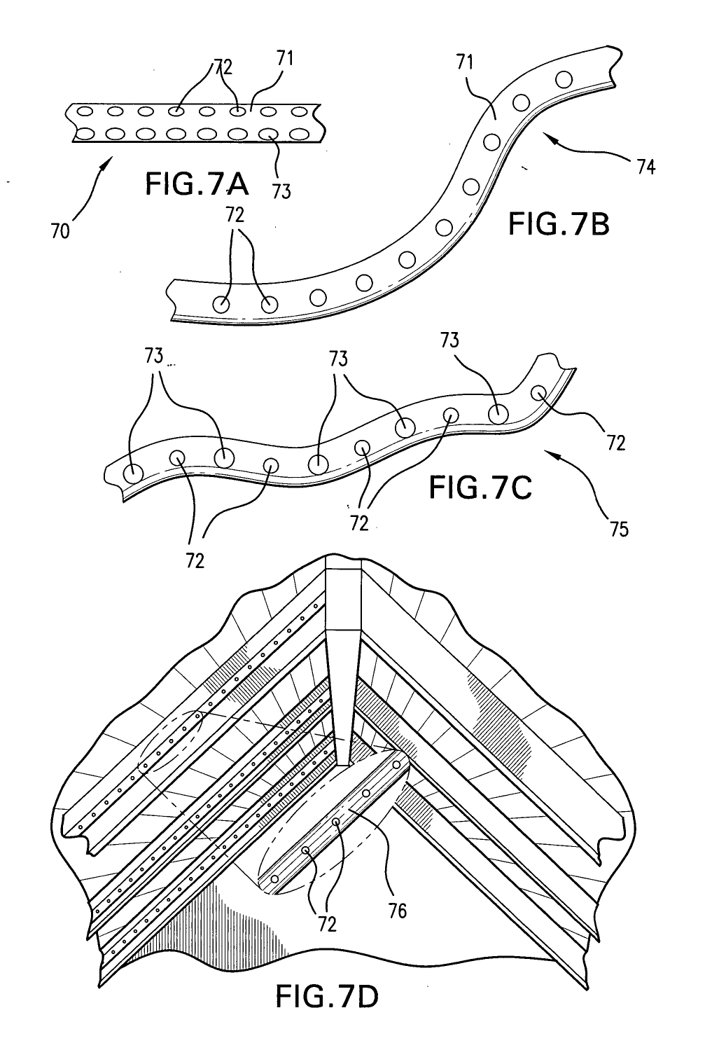

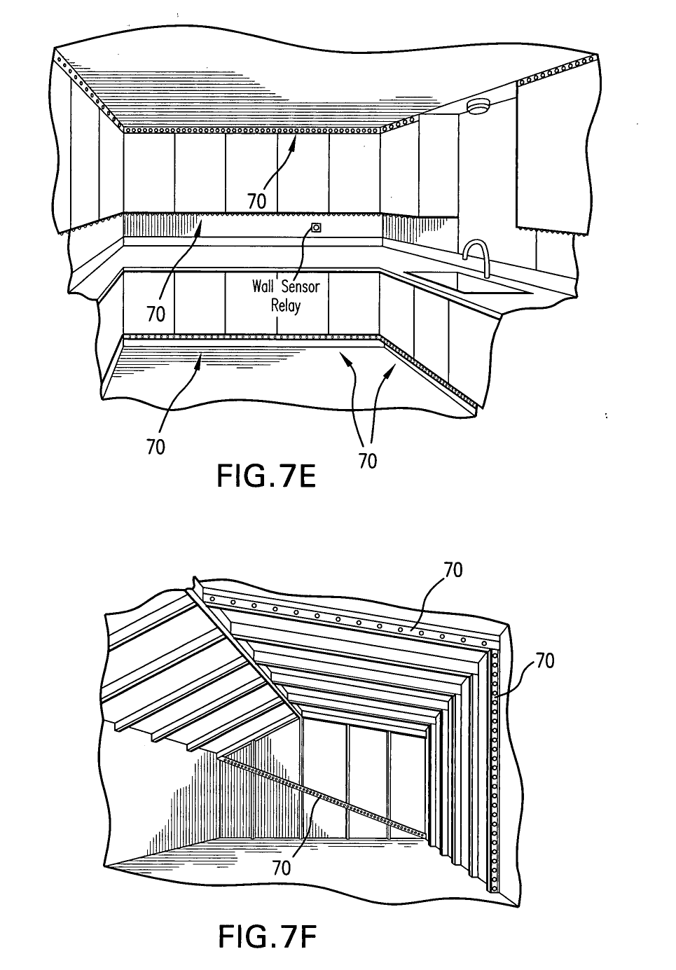

Embodiment VI is illustrated in FIGS. 7A TO 7F and discloses the use of light device 74 including an ultraviolet light source 72 on a housing strip 71 or rope housing 76. As shown in FIG. 7B the light device 74 housing includes only the ultraviolet light source. A preferred application is use of this light device in an attic (FIG. 7F), in crawl spaces, basements, etc., which do not get a lot of people traffic and tend to be musty, and moist which would promote mold, mildew and germ proliferation.

As shown in FIG. 7A the strip may also include a white light source 73. The switches as defined in the other embodiments would also apply to this embodiment. The ultraviolet light source would be activated when no one was present. The ultraviolet light and the white light source may be arranged in parallel rows as shown in FIG. 7A or in a single row with alternating UV and white lights as shown in FIG. 7C.

FIG. 7D illustrates an ultraviolet rope or strip lights for use along framing, cabinets, baseboards, basements and attics. Ultraviolet LED bulbs are placed along the length of the strip or tape and are preferably used along attic rafters or in basements and crawl spaces to retard and prevent mold growth and formation. This embodiment can also be used along cabinet and architectural baseboards and trims in basements with or without white light LEDS as desired.

Switches may be as in previous versions or with manual on/off overrides or exclusively manual on off switches only as desired.

Embodiment VII is illustrated in FIG. 1 and shows a bimodal incandescent device with two filaments inside of one bulb housing, one ultraviolet 1 and one white light source 2; plastic or metal housing 3 and male screw in component 14. The switches used with this device are the same as described with Embodiment I.

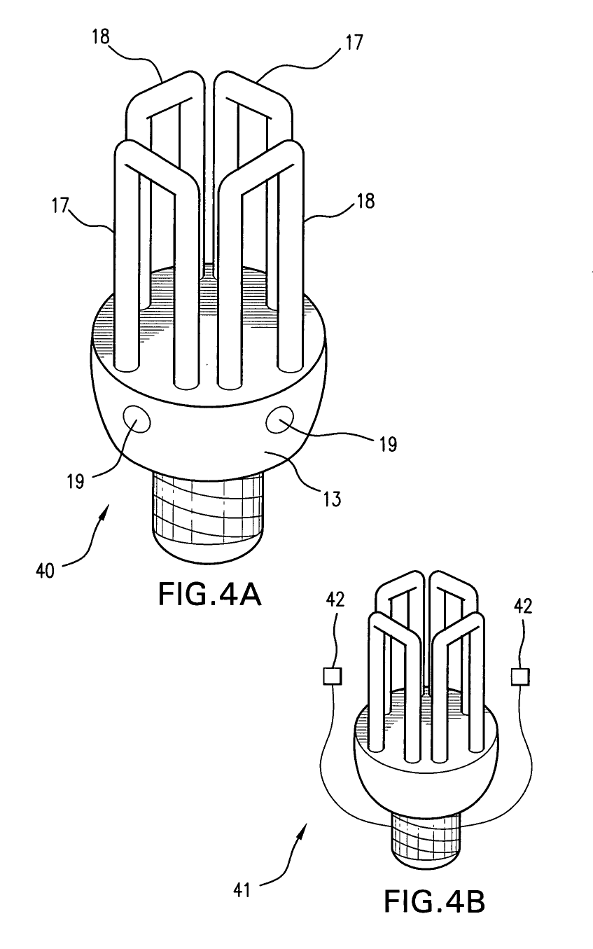

Embodiment VII is illustrated in FIG. 4 and shows a bimodal fluorescent device with one fluorescent bulb with four, or more, fluorescent loops, preferably two as a white light source 18 and two as an ultraviolet source 17 and metal or plastic housing. The switches used with this device are the same as described previously.

In the housing when a person enters the room, the ultraviolet light, two of the four glass loops, are shut off, and the white light is turned on. When no body heat and no movement is detected in the room after a chosen delay period, preferably 1 to 3 minutes or more, the ultraviolet germicidal light source, two of the four glass loops, is turned on, thus cleansing and sanitizing the room.

FIG. 4B illustrates this embodiment where the switches are outside the housing and have sensors 42 to detect motion or heat.

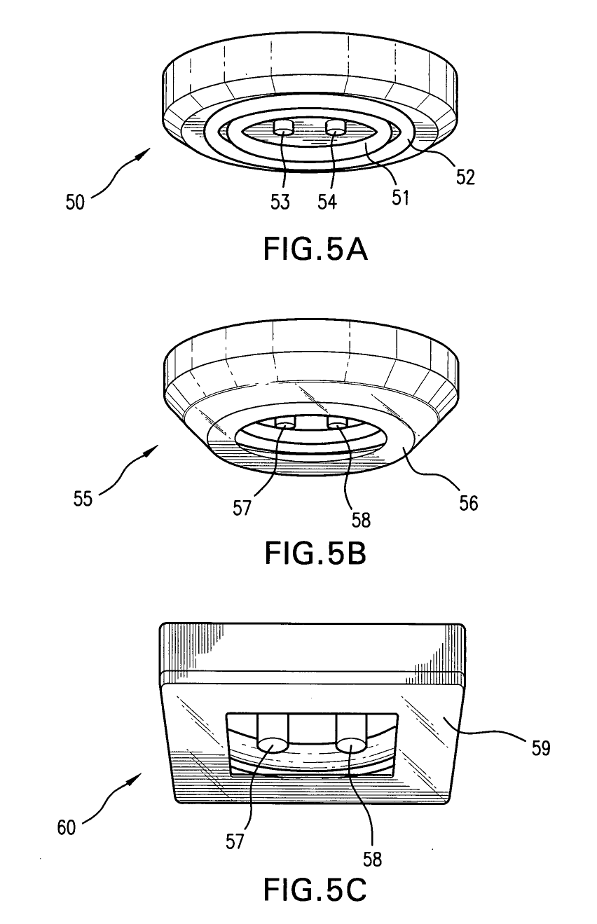

FIG. 5A illustrates a recessed lighting embodiment of the invention where the white light 51 is an outer circular bulb and the UV light 52 is an inner circular bulb. The locations of these bulbs can be interchanged without changing the device. The infrared heat sensor switch 53 and the movement switch 54 are in the center of the inner bulb. The locations of these switches can be interchanged or even removed from the device. FIGS. 5B and 5C are illustrations of the various housing devices for the invention, showing a circular cover 56 and square cover 59 respectively. Both these covers contain openings for the infrared switch 57 and the movement switch 58.

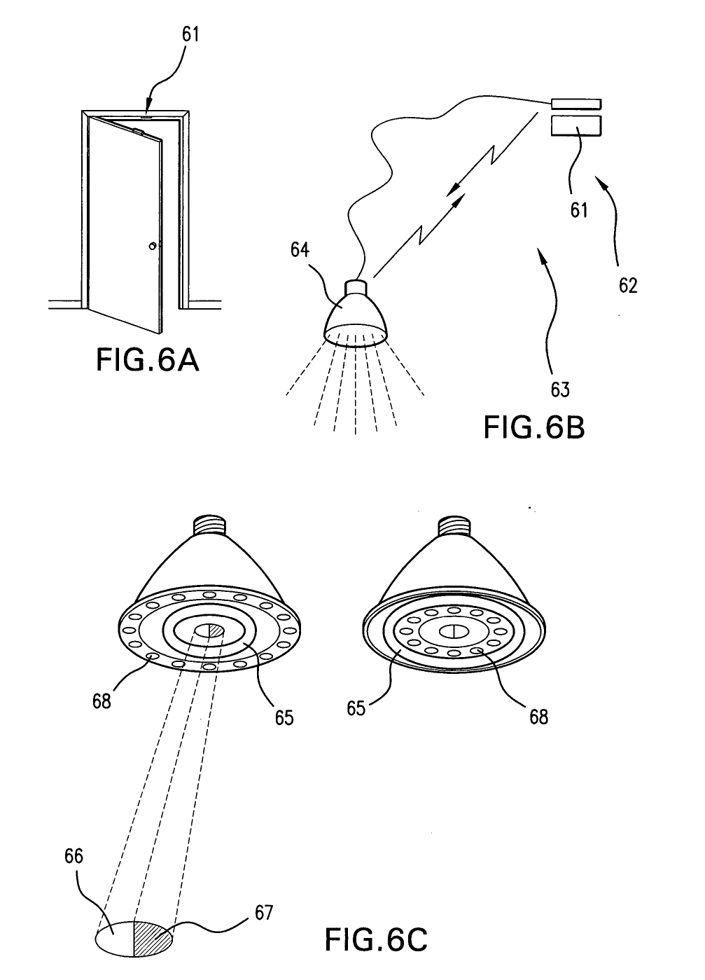

FIGS. 6A to 6C illustrate the process and sterilizing and cleansing system of the invention. The process for sterilizing and cleansing a surface according to the invention is to first provide a light device comprised of an ultraviolet light source and an additional light source; wherein said additional light source is selected from the group consisting of a white light fluorescent and incandescent light. The ultraviolet light source is activated to sterilize and cleanse the surface in view, while the additional light source is deactivated. Deactivating the ultraviolet light source which initializes the additional light source for conventional uses.

The system for sterilizing and cleansing a surface includes a light device comprised of an ultraviolet light source 68 and an additional light source 65; wherein said additional light source is selected from the group consisting of a white light fluorescent and incandescent light. A switch selected from the group consisting of infrared, movement, electric eye, manual, contact switches, auto or semi-automatic, hard wired, pre- and after market versions. As illustrated in FIG. 6C an infrared switch 66 and motion switch 67 are shown. FIG. 6A illustrates a contact switch 61 and FIG. 6B illustrates a natural light source 64 a hard wire or wireless relay 62 and a UV light source 63. Although these switches are illustrated in the drawings others can be used. The switch controls the activation and deactivation of the ultraviolet light source and the additional light source such that they are not both on at the same time.

While the premises of this invention is constant, the configuration of the components, the number, type and strength of light sources (LEDS, fluorescent, incandescent, ultraviolet and white), may vary. The nature of the switches (infrared, movement, electric eye, manual, contact switches, auto or semi-automatic, hard wired, pre- and after market versions) may vary.

The variations provide and allow for switches to be separate and outside of the light unit itself. Placement outside the unit can be on the wall, wall receptacle, ceiling, floor, door, etc.

The sterilization of the designated areas is accomplished via incandescent fluorescent or LED's or any combination or variation of these. Sterilization times and kill-times may vary from continuously on to pulse or flash on sterilization and depend on the desired task and application.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A light bulb comprised of an ultraviolet filament and a white light filament contained within a single outer covering.

2. The light bulb according to claim 1, further containing a housing unit.

3. The light bulb according to claim 2, further comprising a switch within said housing unit.

4. The light bulb according to claim 2, further comprising a switch outside of said housing unit.

5. A light device comprised of an ultraviolet light source and an additional light source; wherein said additional light source is selected from the group consisting of a white light fluorescent and incandescent light.

6. The light device according to claim 5, further containing a housing unit.

7. The light device according to claim 6, further comprising a switch within said housing unit.

8. The light device according to claim 6, wherein said ultraviolet light source are light emitting diodes and are contained within said housing and said additional light source is an incandescent or fluorescent bulb.

9. The light device according to claim 7, wherein said switch is selected from the group consisting of electric eye, infrared, a movement sensor, manual, automatic, hardwired, pre- or semi-automatic and after market.

10. The light device according to claim 6, further comprising a switch outside of said housing unit; wherein said switch is selected from the group consisting of electric eye, infrared, a movement sensor, manual, automatic, hardwired, pre- or semi-automatic and after market.

11. The light device according to claim 5, wherein said ultraviolet light source is a circular outer bulb and said additional light source is a circular inner bulb.

12. The light device according to claim 5, wherein said ultraviolet light source is a circular inner bulb and said additional light source is a circular outer bulb.

13. The light device according to claim 5, wherein said additional light source is a circular outer bulb and ultraviolet light emitting diodes are within the center space of said circular bulb.

14. The light device according to claim 5, further comprising a female receptacle in the center of the device.

15. The light device according to claim 14, wherein said ultraviolet light source is a circular outer bulb and an incandescent bulb is screwed into said receptacle.

16. The light device according to claim 5, further comprising at least two ultraviolet light sources and/or at least two additional light sources.

17. The light device according to claim 5, further comprising a cover.

18. The light device according to claim 5, further comprising strip or rope housing, wherein said ultraviolet light source and said additional light source are secured along this housing.

19. A process for sterilizing and cleansing a surface comprising:
providing a light device comprised of an ultraviolet light source and an additional light source; wherein said additional light source is selected from the group consisting of a white light fluorescent and incandescent light;
activating the ultraviolet light source to sterilize and cleanse the surface in view, while the additional light source is deactivated;
deactivating the ultraviolet light source which initializes the additional light source for conventional uses.

20. A system for sterilizing and cleansing a surface comprising:
a light device comprised of an ultraviolet light source and an additional light source; wherein said additional light source is selected from the group consisting of a white light fluorescent and incandescent light; and
a switch selected from the group consisting of infrared, movement, electric eye, manual, contact switches, auto or semi-automatic, hard wired, pre- and after market versions;
wherein said switch controls the activation and deactivation of the ultraviolet light source and the additional light source such that they are not both on at the same time.

21. A light device comprised of an ultraviolet light source secured along a housing consisting of either a tape strip or rope.

22. The light device according to claim 22, further comprising an additional light source selected from the group consisting of a white light fluorescent and incandescent light.
